(19) United States
(12) Patent Application Publication
(10) Pub. No.: US 2025/0290122 A1
(43) Pub. Date: Sep. 18, 2025

(54) Typing and Assembling Discontinuous Genomic Elements

(71) Applicant: Ludwig Institute for Cancer Research Ltd, Zurich (CH)

(72) Inventors: Bing Ren, La Jolla, CA (US); Jesse Dixon, La Jolla, CA (US); Siddarth Selvaraj, La Jolla, CA (US)

(21) Appl. No.: 19/223,784

(22) Filed: May 30, 2025

Related U.S. Application Data
(63) Continuation of application No. 15/763,577, filed on Mar. 27, 2018.
(60) Provisional application No. 62/234,329, filed on Sep. 29, 2015.

(51) Int. Cl. C12Q 1/6841; C12Q 1/68; C12Q 1/6869; C12Q 1/6876; G16B 20/00; G16B 20/20; G16B 20/40; G16B 30/00; G16B 30/20; G16B 45/00

(52) U.S. Cl. CPC C12Q 1/6841; C12Q 1/68; C12Q 1/6869; C12Q 1/6876; G16B 20/00; G16B 20/20; G16B 20/40; G16B 30/00; G16B 30/20; G16B 45/00; C12Q 2600/172

(57) ABSTRACT

This invention relates to methods and kits for typing and assembling discontinuous genomic elements.

### CROSS REFERENCE TO RELATED APPLICATION

This application is a Continuation of U.S. patent application Ser. No. 15/763,577 filed Mar. 27, 2018, which is national stage entry of International Application No. PCT/US2016/53943 filed Sep. 27, 2016, which claims priority to U.S. Provisional Application No. 62/234,329 filed Sep. 29, 2015. The contents of these applications are all incorporated herein by reference in their entireties.

### FIELD

This invention generally relates to the fields of genetics, molecular and cell biology and, in particular, relates to methods and kits for typing and assembling discontinuous genomic elements and diploid sequencing.

### BACKGROUND OF THE INVENTION

Current Short-read sequencing generates genomic data with poor contiguity and consequently limits de novo assembly of genomes and deconvolution of diploid haplotypes. In the context of typing, each organism has a defining set of chromosomes that contain all of its genetic information. Normal human somatic cells for example are diploid and have two sets of chromosomes, i.e., a paternal set of chromosomes and a maternal set of chromosomes in each nucleus. Within each individual, these two sets of chromosomes have different nucleotide sequences at multiple loci. Understanding the true genetic makeup of an individual requires delineation of the maternal and paternal copies, or haplotypes, of the genetic material. There is a need for tying or diploid sequencing of various genomic elements (e.g., genes and exons) in the genome. While approaches exist for haplotyping entire diploid genomes (Selvaraj et al NBT 2013 December; 31 (12): 1111-8) or targeted loci (Selvaraj et al, BMC Genomics 2015 Nov. 5; 16:900), approaches for haplotyping dis-contiguous genomic elements into a chromosome-span haplotypes are lacking.

### SUMMARY OF INVENTION

This invention addresses the aforementioned unmet need by providing a method and a kit for reconstructing and typing discontinuous genomic elements the whole chromosome or genome level. By exploiting the fact that proximity-ligation experiments capture 3D organizations of genomic elements of interest and because 3D information is long-range information of genomic elements, the method and kit disclosed herein can genotype exons and link all exons into a single chromosome-spanning haplotype.

In one aspect, the invention provides a method for typing and assembling discontinuous genomic elements. The method includes (i) obtaining a plurality of genomic DNA fragments or a genomic sequence data of one or more chromosomes; (ii) obtaining a plurality of element sequence reads for the elements (e.g., exonic sequence reads) from the genomic DNA fragments or the genomic sequence data, and (iii) assembling the plurality of the element sequence reads (such as exonic sequence reads) to construct a long-range or chromosome-span haplotype for the one or more chromosomes. As disclosed herein, the assembling can be carried out using a maxcut algorithm.

In some embodiments, the plurality of genomic DNA fragments can be obtained using a technique selected from the group consisting of Hi-C, 3C, 4C, 5C, TLA, TCC, and in situ Hi-C. For example, the plurality of genomic DNA fragments can be obtained by a process including (i) providing a cell that contains a set of chromosomes having genomic DNA; (ii) incubating the cell or the nucleus thereof with a fixation agent for a period of time to allow crosslinking of the genomic DNA in situ and thereby forming crosslinked genomic DNA; (iii) fragmenting the crosslinked genomic DNA; (iv) ligating the crosslinked and fragmented genomic DNA to form a proximally ligated complex; (v) shearing the proximally ligated complex to form proximally-ligated DNA fragments; and (vi) obtaining a plurality of the proximally-ligated DNA fragments to form a library thereby obtaining the plurality of genomic DNA fragments. Examples of the discontinuous genomic elements can be selected from the group consisting of genes, exons, introns, untranslated regions, protein domain-coding sequences, gene fusions, transcription factor binding sites, promoters, enhancers, silencers, conserved elements, miRNA-coding sequences, miRNA binding sites, splice sites, splicing enhancers, splicing silencers, structure variants, common SNPs, UTR regulatory motifs, post translational modification sites, common elements, and any other elements of interest.

In the method mentioned above, the fragmenting step can be carried out by restriction enzyme digestion with one or more enzymes. Preferably, the digestion can be carried out with two or more different enzymes. The enzymes can be a 4-cutter or 6-cutter. In one example, at least one enzyme can be selected from the group consisting of DpnII, MboI, HinfI, HindIII, NcoI, XbaI, and BamHI.

In the method mentioned above, the plurality of sequence reads (such as exonic sequence reads) can be obtained from the genomic DNA fragments by a process comprising: (i) hybridizing the plurality of genomic DNA fragments with a set of probes to form a hybridization mixture; (ii) separating probes that are hybridized to isolate a subgroup of the genomic DNA fragments, and (iii) sequencing the isolated genomic DNA fragments to generate a plurality of sequence reads thereby obtaining the plurality of sequence reads (such as exonic sequence reads). Before the sequencing step, this method can further comprise amplifying the isolated genomic DNA fragments if a large quantity of the captured DNA is needed.

In some examples, to obtain exonic sequences, the probes have sequences complementary to exonic sequences in the one or more chromosomes, and they can be cDNA probes or RNA probes.

To facilitate isolation, each probe can comprise an affinity tag. Examples of the affinity tag include a biotin molecule and a hapten. The separating step can include contacting the hybridization mixture with an agent that binds to the affinity tag. Examples of the agent include an avidin molecule, or an antibody that binds to the hapten or an antigen-binding fragment thereof. In some embodiments, the probes can be attached to a support, such as a microarray. In that case, the support can include a planar support having one or more substrate materials selected from glass, silicas, metals, Teflon, and polymeric materials. Alternatively, the support can include a mixture of beads, each bead having one or more probes bound thereto and the mixture of beads can include one or more substrate materials selected from nitrocellulose, glass, silicas, Teflon, metals, and polymeric materials.

The above-described method can further include a step of isolating the cell nucleus from the cell before the incubating step, or purifying genomic DNA before the fragmenting step. The fixation agent can be formaldehyde, glutaraldehyde, formalin, or a combination thereof. The sequencing step can be carried out using NGS. Each sequence read can be at least 75 bp (e.g., 100 bp, 150 bp, 200 bp, or 250 bp) in length and for each chromosome the library contains at least 10× (e.g., 20×, 30×, 40×, or 50×) sequence coverage.

The above-described method can be used for typing various genomic elements, including but not limited to exome haplotyping, of any chromosomes of a cell from an organism, and diploid sequencing. It can be used to type (e.g., haplotype) or sequence any eukaryote, including a fungus, a plant, or an animal such as a mammal or a mammalian embryo (e.g., a human or a human embryo).

In a second aspect, the invention provides a kit for carrying out the method described above, including but not limited to exome haplotyping one or more chromosomes. The kit includes a fixation agent, one or more restriction enzymes, a ligase, a set of probes that are complementary to sequences of the discontinuous genomic elements (such as exonic sequences) in the one or more chromosomes, and are labeled with an affinity tag, and an agent capable of binding to the affinity tag. The kit can further include one or more components selected from the group consisting of a cell lysis buffer, one or more restriction enzyme reaction buffers, a hybridization buffer, extension nucleotides, a DNA polymerase, a protease, adaptors, blocking oligonucleotides, an RNAse inhibitor, and reagents for sequencing. At least one of the extension nucleotides can be labeled with an affinity tag.

The details of one or more embodiments of the invention are set forth in the description below. Other features, objectives, and advantages of the invention will be apparent from the description and from the claims.

### BRIEF DESCRIPTION OF THE DRAWINGS

FIGS. 1a and 1b are two sets of diagrams showing (FIG. 1a) an exemplary Whole-Exome Haplotyping experimental design and (FIG. 1b) a computational strategy to link proximal and distal exonic variants with the help of short-range and long-range chromatin interaction data into a single haplotype block.

FIGS. 2a and 2b are diagrams showing that in situ Hi-C datasets generate more usable data when compared to conventional Hi-C dataset: (FIG. 2a) fraction of long-range (>20,000) and short-range Cis (Intra-chromosomal) fragments, and (FIG. 2b) fraction of trans fragments.

FIGS. 3a, 3b, 3c, 3d, and 3e are a set of diagrams showing whole-exome proximal ligation libraries can generate chromosome-span haplotypes at difference read lengths: (FIG. 3a) 50 bp, (FIG. 3b) 75 bp, (FIG. 3c) 100 bp, (FIG. 3d) 150 bp, and (FIG. 3e) 250 bp.

FIGS. 4a, 4b, and 4c are (FIG. 4a) a diagram showing single-enzyme and multi-enzyme Whole-exome HaploSeq, (FIG. 4b) a table showing single-enzyme and multi-enzyme Whole-exome HaploSeq using NcoI and XbaI, and (FIG. 4c) four tables showing (c-i) comparison of performance by NcoI and multi-enzyme, (c-ii) results of whole-genome genotyping using NcoI, (c-iii) results of whole-genome genotyping from using Multi-enzyme, (c-iv) results of whole-genome genotyping combined dataset.

FIGS. 5a and 5b are two tables showing evaluation metrics of Whole-exome HaploSeq: (FIG. 5a) phasing results across all the haplotype block and (FIG. 5b) phasing results across the block with the most variant phased (MVP).

FIG. 6 is a diagram showing impact of restriction enzyme choice on read coverage.

### DETAILED DESCRIPTION OF THE INVENTION

This invention is based, at least in part, on an unexpected discovery that reconstructing whole genome haplotypes at chromosome-span level can be achieved by targeting sub-regions of the genome, such as one or more sets of discontinuous genomic elements including but not limited to exons, and by exploiting their three-dimensional organization.

It is challenging to generate high-quality haplotype phasing for a diploid genome in a practical and scalable manner. Previously, a so-called HaploSeq method was developed to generate chromosome wide haplotypes with the use of proximity ligation approach (Selvaraj et al. Nat Biotechnol 31, 1111-8 (2013) and WO2015010051). However, HaploSeq requires a large number of sequence reads to phase a human genome, which is prohibitively expensive using today's sequencing technologies.

Disclosed herein in one example is a new phasing method that achieves whole genome phasing and generates chromosome-span haplotyping by specifically targeting a small fraction (less than 2%) of the genome, for example, the exomes (or protein-coding regions or other discontinuous genomic elements as described herein) of the genome. Specifically, inventors used proximity-ligation and capture sequencing to enable analyses of dis-contiguous elements of genome. For instance, exome capture of proximity-ligation libraries allows exome proximity-ligation datasets (Exome PL) that has several applications: De novo assembly of exome, exome genotyping, chromosome-span haplotyping of exome, gene fusion analyses, exonic structural variant analyses, understanding three-dimensional (3D) organization of exomes, etc.—enabling typing and assembling of exomes. Similarly to exome capture, other types of dis-contiguous elements such as set of common variants in the genome, set of cancer or disease-specific genes, etc. can be captured, typed and assembled.

In some embodiments, an exome-focusing method, which is referred as Whole-Exome

HaploSeq, incurs less than 10% of the cost of HaploSeq, and provides exome sequences at the same time. Phasing all exonic regions of the genome into a single haplotype structure has a wide variety of applications in precision medicine, including, but not limited to, non-invasive pre-natal diagnostic tests (NIPT), and disease gene discovery in cases of compound heterozygosity. See e.g., Bianchi, D. W. Nat Med 18, 1041-51 (2012), Browning et al. Genetics 194, 459-71 (2013), Tewhey et al. Nat Rev Genet 12, 215-23 (2011), Kitzman, et al. Sci Transl Med 4, 137ra76 (2012), and Browning et al. Am J Hum Genet 81, 1084-97 (2007).

While certain embodiments disclosed herein focus on whole-exome HaploSeq, the targeted approach described herein can be used to target other features or elements of the genome. For example, one can design probes to target common variants in the genome and achieve Common-variant HaploSeq, using the same experimental and computational principles illustrated in this application. Together, by targeting sub-regions of the genome and by exploiting their three-dimensional organization, one can obtain chromosome-spanning haplotypes for these variants.

### Haplotyping and Reconstruction

Haplotype reconstruction, also known as “haplotype phasing”, is the use of DNA sequencing data to group variant alleles that are inherited from the same parent. This grouping is called a haplotype block. See Browning et al. Am J Hum Genet 81, 1084-97 (2007). The utility of obtaining haplotype information in an individual can be several folds. First, phasing information of exons is crucial to predict disease risks for compound mutations in a gene (Tewhey et al. Nat Rev Genet 12, 215-23 (2011)). Second, knowledge of haplotype structures is useful clinically for pre-natal non-invasive fetal sequencing (Kitzman, et al. Sci Transl Med 4, 137ra76 (2012)). In addition, haplotypes are also useful for predicting outcomes for donor-host matching (HLA/KIR matching) in organ transplantation and for understanding graft rejection tolerance mechanisms (Petersdorf et al. PLoS Med 4, e8 (2007)). Further, haplotypes are useful in understanding “allelic imbalances” in gene expression, DNA methylation, and protein-DNA interactions, which are known to influence disease susceptibility (Kong, A. et al. Nature 462, 868-74 (2009), International Consortium for Systemic Lupus Erythematosus, G. et al. Genome-wide association scan in women with systemic lupus erythematosus identifies susceptibility variants in ITGAM, PXK, KIAA1542 and other loci. Nat Genet 40, 204-10 (2008), and Hindorff et al. Proc Natl Acad Sci USA 106, 9362-7 (2009)). Haplotypes, and in particular chromosome-span haplotypes, can also help in constructing ancestry and in delineating population migration patterns (International HapMap, C. et al. Nature 449, 851-61 (2007), Genomes Project, C. et al. A map of human genome variation from population-scale sequencing. Nature 467, 1061-73 (2010), and Genomes Project, C. et al. An integrated map of genetic variation from 1,092 human genomes. Nature 491, 56-65 (2012)). Taken together, obtaining haplotype information is important for clinical and biomedical advances in human genetics.

Several methods, including HaploSeq, chromosome sorting or segregation, sperm genotyping or parent-child trio sequencing, are capable of generating chromosome-spanning haplotypes. See, e.g., Selvaraj et al. Nat Biotechnol 31, 1111-8 (2013), Genomes Project, C. et al. A map of human genome variation from population-scale sequencing. Nature 467, 1061-73 (2010), Genomes Project, C. et al. An integrated map of genetic variation from 1,092 human genomes. Nature 491, 56-65 (2012), Ma et al. Nat Methods 7, 299-301 (2010), Fan, et al. Nat Biotechnol 29, 51-7 (2011), Yang et al., Proc Natl Acad Sci USA 108, 12-7 (2011), and Kirkness et al., Genome Res 23, 826-32 (2013). However, chromosome-scale haplotypes are expensive to generate and therefore have limited utility for practical purposes.

Disclosed herein in one example is a method that targets all genes (or exons) of the genome and reconstruct chromosome-span haplotypes of phased whole exome. An important and surprising achievement of the method is the ability to reconstruct chromosome-span haplotypes from analysis of only the exome. As exons are randomly distributed across the chromosome, it has heretofore been mathematically very difficult to link all exons into a single haplotype structure. Specifically, the discontinuous nature of exons makes it very challenging to assign single haplotype phase for all exons. Consequently, conventional chromosome-span haplotype methods, which cannot handle such discontinuous nature of exons, cannot phase them into a single haplotype.

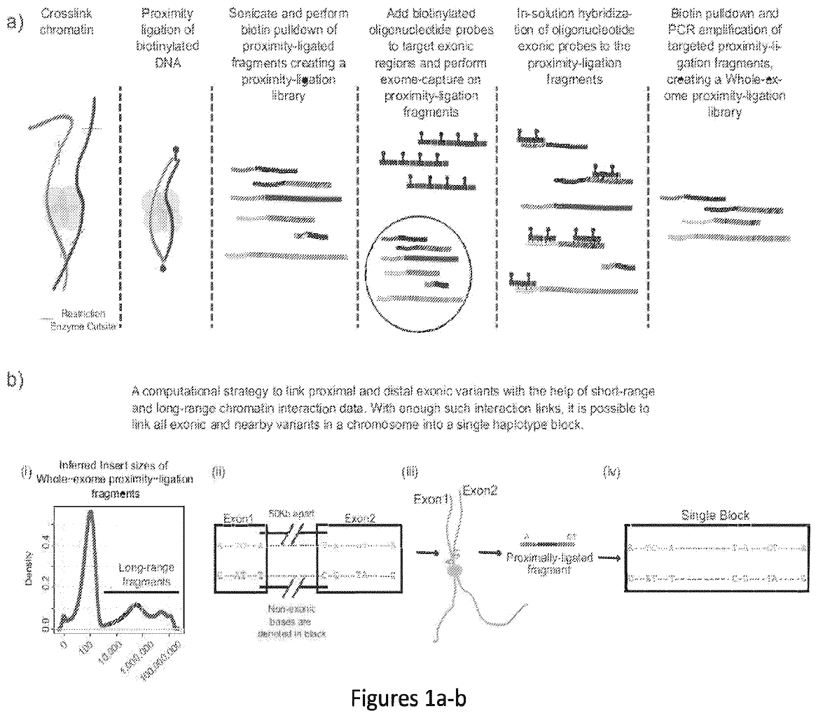

As disclosed herein, this problem was solved by using novel experimental and computational strategies. Shown in FIG. 1 is a design of an exemplary method of this invention, which focuses on the development of genotyping and whole-exome haplotyping. In particular, the designs exploits the long-range fragments generated by proximity-ligation experiments (FIGS. 1a and 1b-i) to link spatially proximal exons in to a single haplotype structure (FIG. 1b). With sensitive exome capture methodologies, enough sequencing coverage and novel computations tools, all the exons in a chromosome can be linked into a single haplotype.

In one example, one can first crosslink the chromatin with formaldehyde or other crosslinking agents. The chromatin can be then digested with one enzyme or a set of different restriction enzymes of choice and the spatially proximal chromatin can be ligated and sonicated resulting in a library of proximity-ligation fragments. Exome capture can be then used to target and capture exonic proxmity-ligated fragments. This results in a whole-exome proximity-ligation library. FIG. 1b shows an insert-size distribution of such a whole-exome proximity-ligation library. The library consists of a mixture of short, intermediate and long-range interactions that will help to link proximal as well as distal exonic variants (FIG. 1-b-i). As illustrated in FIG. 1b-ii, exon1 and exon2 are 50-kb apart; variants within each exon are linked by short-range chromatin interactions, resulting in two exon blocks (FIG. 1-b-ii). As variants in exon1 and 2 are spatially proximal but linearly distal-50 kb apart, they can be linked by a long-range interaction (FIG. 1-b-iii) and consequently converging the two exon blocks into one block. With enough data, such smaller exon blocks can be linked into a chromosome-span single haplotype structure.

As shown in the examples below, this above-described Whole-Exome HaploSeq, allowed effective capture and three-dimensional organization of exons. In addition, exons were successfully linked based on the whole-exome HaploSeq data by using an innovative graph-based computational algorithm, where exons are considered as edges in a graph.

### Proximity-Ligation

In the design shown in FIG. 1a, a proximity-ligation based method is used for DNA sequencing library preparation, followed by oligonucleotide-based exome capture and high throughput DNA sequencing. The proximity-ligation can be carried out using the Hi-C method in the manner described in Lieberman-Aiden, et al. Science 326, 289-93 (2009), the content of which is incorporated herein by reference.

In one example, the initial steps can be identical to the HaploSeq method as described in Selvaraj et al. Nat Biotechnol 31, 1111-8 (2013) and WO2015010051. More specifically, cells can be cross-linked with a crosslinking agent to preserve protein-protein and DNA-protein interactions. This can be carried out at room temperature for 10-30 minutes with 1-2% of formaldehyde. The cells can be then harvested by centrifugation and can be stored at −80° C. The cells can be lysed in a hypotonic nuclear lysis buffer, and then washed with a 1× concentration of buffer for the restriction enzyme of choice (e.g., from New England Biolabs). The cells can be digested for 1 hour to overnight with 25 U to 400 U of enzyme, depending upon the enzyme used. Four-base cutting enzymes benefit from short digestions with less amount of enzyme (e.g., 1 hour with 25 U), whereas six-base cutting enzymes can use longer digestions with larger amounts of enzyme. The ends of DNA can be repaired with Klenow polymerase in the presence of dNTPs, one of which (e.g., dATP) can be covalently linked to biotin. The sample can be then ligated in the presence of T4 DNA ligase for 4 hours. The sample can be then digested overnight in the presence of Proteinase K at 65° C. to reverse cross-links and degrade protein. The DNA can be then isolated using, e.g., a series of phenol-chloroform extractions and ethanol precipitations. After the purified DNA is isolated, it can be sonicated on a Covaris or Bioruptor machine. The DNA can be then end repaired, and A-tailed according to standard library pre-preparation methods. The A-tailed DNA can be then bound to streptavidin coated beads to isolate the biotinylated, ligated DNA fragments. The beads can be washed to remove non-specific, unbiotinylated DNA fragments. Adaptors can be then ligated to the Illumina Tru-Seq adaptor set using Quick DNA Ligase. 1 μL of the sample can be then diluted 1:1000 and the concentration can be tested by qPCR against known standard (KAPA). The sample can be then amplified by PCR to obtain sufficient material, which in general means a total of 750 ng of sample across all libraries to be captured. The PCR amplified libraries can be purified using AMPure beads, and the final concentration can be again tested by making a 1:1000 dilution and testing against known standards by qPCR (KAPA).

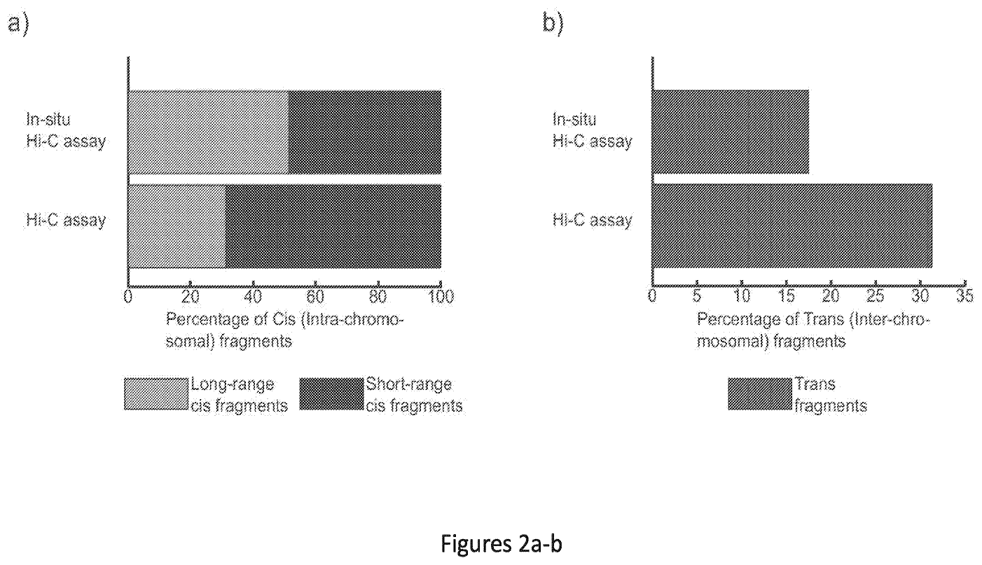

Although the Hi-C protocol is used as the proximity-ligation protocol in the figures, variations such as 3C, 4C, 5C, TLA, TCC, in situ Hi-C and other protocols, can also be used for methods disclosed here in, such as the Whole-exome HaploSeq. Details of these protocols can be found in Lieberman-Aiden, et al. Science 326, 289-93 (2009), Dekker et al., Science 295, 1306-11 (2002), van de Werken et al. Methods Enzymol 513, 89-112 (2012), Simonis et al. Nat Methods 6, 837-42 (2009), Dostie et al. Nat Protoc 2, 988-1002 (2007), Nora et al. Nature 485, 381-5 (2012), Sanyal et al., Nature 489, 109-13 (2012), de Vree, P. J. et al. Nat Biotechnol 32, 1019-25 (2014), Kalhor et al. Nat Biotechnol 30, 90-8 (2012), and Rao et al. Cell 159, 1665-80 (2014). All of these references are incorporated herein by reference in their entireties. For example, in-situ Hi-C(Rao et al. Cell 159, 1665-80 (2014)) datasets may be useful for HaploSeq as they generate more long-range fragments (FIG. 2a) and fewer trans-interactions (or inter-chromosomal interactions, which are of less utility for HaploSeq, FIG. 2b) when compared to conventional Hi-C (Lieberman-Aiden, et al. Science 326, 289-93 (2009)). Regardless, by using Hi-C despite its “noisy” data is an important proof of principle that the use of the Hi-C may be sufficient for this purpose.

### Restriction Enzyme Digestion

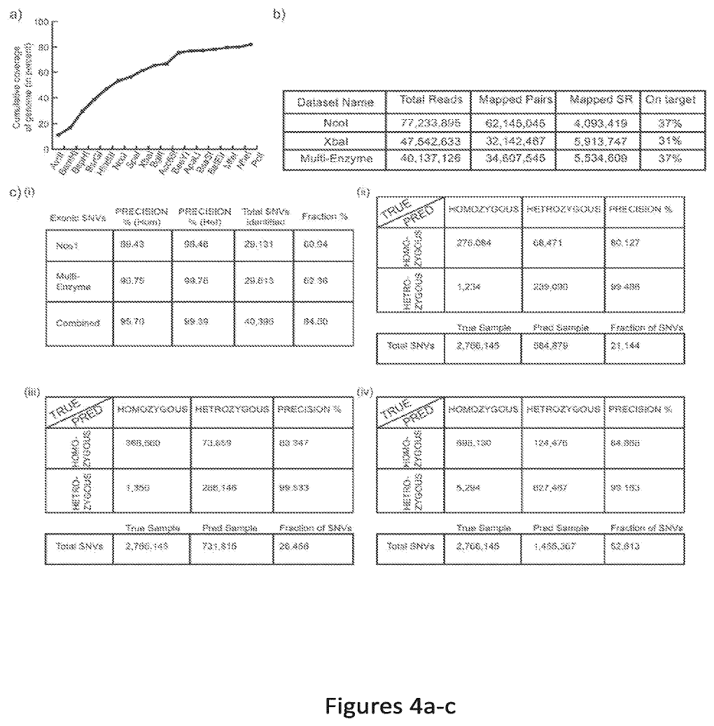

The proximity-ligation protocols described above involve a restriction enzyme digestion prior to proximity-ligation of chromatin. As most of the sequencing reads are distributed near (˜500 bp) the restriction enzyme cut-site, the choice of enzyme used can impact the results. For example, elements (such as exons) that are distal to the chosen restriction enzyme cut sites are less likely to be captured and consequently haplotype phased. To maximize phasing of all elements or variants, one can use multiple enzymes for chromatin digestion. To this end, any single 6-base cutting restriction enzyme can generate proximity-ligation data that covers 5-10% of the genome, but by using multiple such enzymes in the same experiment, one can cover >80% of the genome (FIG. 4a). In addition, a 4-base cutter enzyme or a set of 4-base cutters can be used instead of 6-base cutting enzymes to further maximize the coverage of the genome.

The method disclosed herein, such as the Whole-Exome HaploSeq procedure, can be performed using any number of restriction enzymes provided that they generate sufficient initial HaploSeq libraries. The issue of enzyme choice does have an effect in terms of the number of bases that are covered and phased. For instance, 6-base cutting enzymes cut every ˜4 kb in the genome, and therefore a relative minority of polymorphisms that could be phased falls close enough to cut sites to be phased. In contrast, 4-base cutting enzymes cut much more frequently, on the order of every 250 bp (on average). In this regard, a much larger percentage of polymorphisms will fall close to enzyme cut sites and therefore have the potential to be phased. This can be important for phasing of rare variants, as the latter steps of the HaploSeq method are based on population based imputation, which does not work well for rare variants.

As shown in Examples 2 and 3 below, utilizing a 4-base cutting enzyme or a mixture of different enzymes led to greater coverage with less sequencing read depth. More specifically, while HaploSeq can be successfully performed using one restriction enzyme, multi-enzyme HaploSeq can generate more uniform distribution of data and consequently higher-resolution HaploSeq. See FIG. 4a. As shown in FIG. 4b, three independent Whole-Exome HaploSeq datasets were generated using enzyme NcoI, XbaI and Multi-Enzyme (NcoI, HindIII, and BamHI). As HaploSeq datasets can be used for genotyping, the inventors called SNVs using these datasets. As shown in FIG. 4c (i), the inventors compared the performance of NcoI, Multi-enzyme as well as a combined dataset (NcoI, XbaI and Multi-enzyme) and it was observed that the each of these datasets generated high-accuracy genotyping for heterozygous and homozygous exonic variants. Of note, inventors compared genotype calls to pre-existing WGS data (referred to as True dataset, International HapMap, C. et al. Nature 449, 851-61 (2007) and Genomes Project, C. et al. A map of human genome variation from population-scale sequencing. Nature 467, 1061-73 (2010).) Further, the exonic genotyping was of high-resolution (>85% of exonic SNVs genotyped in the combined dataset). Because these datasets also can span non-exonic regions, the inventors checked the genotyping capabilities of all variants-exonic and non-exonic. Thus multi-enzyme data can be more useful for genotyping and potentially haplotyping or de novo assembly applications when compared to single enzyme dataset.

### Genomic Element Capture

The next step in the protocol is to capture the amplified Hi-C library. Examples of capture probes include those of Agilent SureSelectXT2 v5 capture library, though one can use any library covering exons or other discontinuous regions (for instance, targeting exons containing restriction enzyme sites, or targeting restriction enzyme sites near sequences of interest, such as exons or regulator regions). The hybridization can be done according to the manufacturer's instructions.

In general, the process for capture of targeted genomic DNA fragments can be as follows: (1) DNA can be obtained from biological samples; (2) the DNA can be fragmented by various means including mechanical, ultrasonic or enzymatic approaches; (3) targeted DNA fragments can be captured selectively by hybridizing DNA fragments with complimentary DNA and/or RNA probes or baits; (4) DNA fragments not bound to the hybridization probes can be washed away first, while DNA fragments bound to the hybridization probes can be eluted in the next step under appropriate conditions; and (5) the captured DNA can be used for downstream applications.

If a larger quantity of the captured DNA is needed, polymerase chain reactions (PCRs) can be performed to amplify the captured DNA fragments by using a pair of universal primers. The universal DNA primers of specifically-designed sequences (also known as adaptors or indexing adaptors) can be ligated to 5′- and 3′-ends of all DNA fragments, after either step (2) or step (4). Alternatively, the adaptors can be attached during step (2) when the extracted DNA is fragmented by, e.g., an adaptor loaded transposase enzyme. Detailed procedures can be found in, e.g., the SureSelect Target Enrichment System™ marketed by Agilent Technologies, Inc. and US 20100029498.

To capture DNA fragments, the hybridization of DNA fragments and complimentary baits/probes takes place either on solid supporting materials or in liquid solutions. This capture step (step 3 in the above described process) is crucial for the entire process. Specificity of the capture is determined by the DNA or RNA sequences of the hybridization baits/probes. These DNA and/or RNA baits/probes must have sequences precisely complementary to the regions of interest in the genomic DNA of the biological samples of interest. Capacity of the capture is determined by a combination of the number and length of different probes available for use in the hybridizations. Longer-length probes require fewer probes to cover the same DNA region for capture. Flexibility of the capture is determined by the way the probes are generated and placed on either solid supporting materials or mixed in liquid solutions. These hybridization DNA and/or RNA baits should have the overall capacity and flexibility to selectively capture all genomic elements of interest, such as exons, or any subsets of exons, or any other desired regions of genomic and other forms of DNA from any biological species.

In one example, 750 ng of the sequencing library can be used and concentrated into a total volume of 3.4 μl. This can be then combined with 6.6 μl of blocking oligos. Blocking oligos that can be used include those marketed by Agilent Technologies Inc. or the IDT xGen blocking oligos (0.3 uL of p5, 0.3 uL of p7, depending on the collection of Illumina TruSeq adaptors used). This can be then combined with the hybridization buffer and the capture probe library and hybridized overnight at 65° C. The next day, the libraries can be washed exactly according to the manufacturer's instructions. 1 μL of the final bead bound library can be then diluted 1:1000 and tested by qPCR against known standards to determine the number of cycles necessary to obtain enough material to sequence. The library can then be sequenced on the Illumina sequencing platform.

Examples of genomic elements that can be used to practice the method disclosed herein include known genes, exons, introns, untranslated regions, protein domain-coding sequences, transcription factor binding sites, promoters, enhancers, silencers, conserved elements, miRNA-coding sequences, miRNA binding sites, splice sites, splicing enhancers, splicing silencers, common SNPs, UTR regulatory motifs, post translational modification sites, common elements and custom elements of interest. Genomic elements can be continuous or discontinuous in a genome of interest. The method disclosed herein can be used to analyze both continuous genomic elements and discontinuous genomic elements. In one example, it is particularly useful to analyze one or more sets of discontinuous genomic elements for diploid sequencing, genotyping, haplotyping or phasing, and genotype-phenotype studies. In some embodiments, examples include one or more sets of common variants, cancer related genes, Mendelian genes, immune genes, rare variants, etc. Examples of cancer related genes include those listed at the website of American Oncology (ASCO), www.cancer.net/navigating-cancer-care/cancer-Society of Clinical basics/genetics/genetics-cancer. Examples of immune genes include those kept and listed at the website of the Immunological Genome Project (ImmGen), www.immgen.org.

The method described herein allows one to type and sequence genomic elements not only at a single-locus level (e.g., the HLA locus), but also at a multi-locus level (e.g., 2, 3, 4, 5, 6, 7, 8, 9, 10, 15, 20, 50, 100 or more loci), at a single chromosome level, at a multi-chromosome level, and at the whole genome level. Accordingly, in preferred embodiments, the method disclosed herein can be used for multi-locus, discontinuous genomic elements. In that case, a substantial portion or all of the genomic elements of interest from at least one entire chromosome or from the entire genome of a subject are typed or sequenced. For that, the hybridization baits/probes have sequences that hybridize to these multi-locus, discontinuous genomic elements. Haplotyping and Reconstruction

The computational-algorithmic aspect of the approach described herein follows similar principles as in Whole-genome HaploSeq, as detailed in Selvaraj et al. Nat Biotechnol 31, 1111-8 (2013) and WO 2015010051, the contents of which are incorporated herein by reference in their entireties. To that end, one can consider heterozygous variants as nodes in the graph and draw edges between nodes when a HaploSeq read supports them. When the data has no errors this graph trivially deconvolutes the maternal and paternal haplotypes. However, the HaploSeq data generally introduces spurious edges and therefore one can use an algorithm based on Maxcut to predict the likely haplotype structure, given the HaploSeq data. Broad aspects of the algorithm are detailed in Bansal et al., Bioinformatics. 2008 Aug. 15; 24 (16): i153-9, the content of which is incorporated by references in its entirety.

Once this algorithm defines the most possible haplotype structure of an individual (initial haplotype), one can use population based linkage disequilibrium (LD) information (for example from the 1000 genomes project) to fill-in phase information of variants not resolved by the initial haplotype prediction. This step is defined as local conditional phasing (LCP) in Selvaraj et al. Nat Biotechnol 31, 1111-8 (2013).

An important difference between Whole-genome HaploSeq and Whole-exome HaploSeq is that in the case of Whole-exome, the heterozygous variants belong mostly to the exonic regions of the genome. As exons occupy about only 1-2% of the genome and are randomly distributed in their genome locations, it was surprising and unexpected that utilizing only exonic variants alone, one can construct a chromosome-spanning haplotype graph, which can later be augmented by LCP. Therefore, rather than utilizing all heterozygous variants from the whole-genome HaploSeq data, one can restrict the initial graph to contain variants from mostly exons. This reduces the cost of Whole-exome HaploSeq and still allows its utility for haplotype applications such as non-invasive prenatal diagnostics.

As described above, the discontinuous element sequence reads, e.g., exonic sequence reads for exome haplotyping, can be obtained by a process including, among others, element capture, before subjecting the data to the algorithm based on Maxcut to obtain the haplotype structure. One can also directly use genomic sequence data obtained without the capture, such as the data generated using Whole-genome HaploSeq as detailed in Selvaraj et al. Nat Biotechnol 31, 1111-8 (2013) and WO2015010051. To that end, one can take whole-genome HaploSeq data (which is represented by paired-end sequencing read), and extract and keep only the data that spans those genomic elements of interest (such as an exonic variant) in at least one end of the paired-end data. This new data now reflects whole-exome HaploSeq.

The above-described assembling can also be carried out using a hidden Markov model (HMM) known in the art to obtain the haplotype structure. See e.g., Browning et al., Nature Reviews Genetics 12, 703-714 October 2011, US20140045705, and US20130316915. The contents of these references are incorporated by reference in their entireties.

In the above-described approaches, one can construct the graph of heterozygous variants spanning genomic elements of interest (such as exons) and determine whether this graph will have enough edges (or reads) to link all the variants into a single chromosome-spanning haplotype. This is defined by the metric “completeness.” Another metric-“resolution”-defines the number of variants the chromosome-spanning complete graph touches. These two metrics allow one to evaluate the performance of the haplotype reconstruction or haplotype phasing.

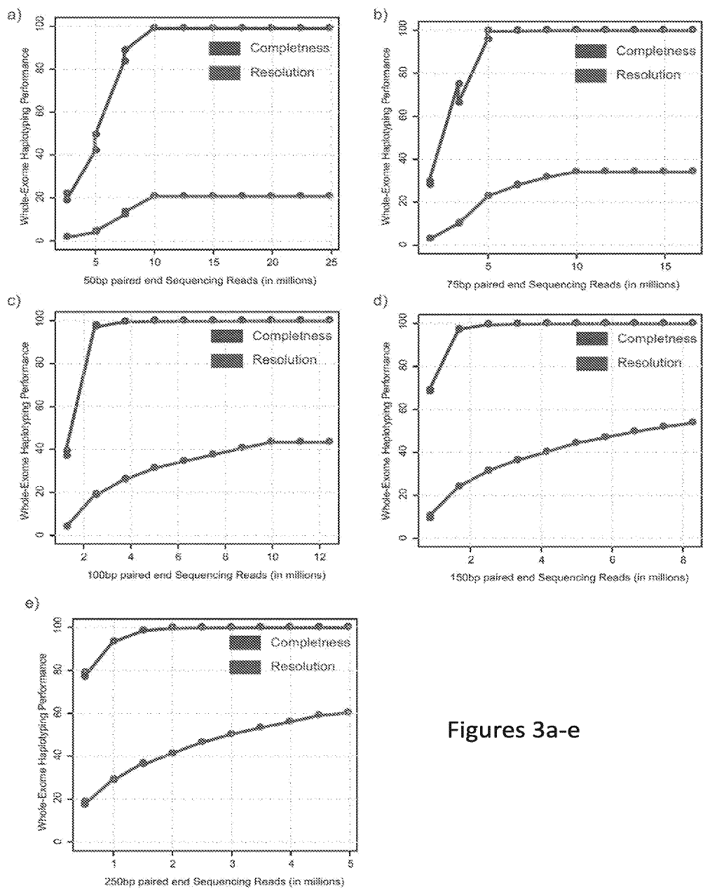

As described in the examples below, several parameters such as read length (FIGS. 3 a-e) and sequencing depth can be changed. Overall, as the read length increases (FIGS. 3a-e), less and less amounts of sequencing reads will be enough to generate complete chromosome-spanning haplotypes with high resolution (20-60% depending on read length and sequencing depth).

The novel strategy described herein allows one to link all genomic elements of interest (such as exons) and phase them together into a single chromosome-span haplotype. For example, with this method for chromosome-scale whole-exome haplotyping, several improvements were achieved over the conventional HaploSeq approach. First, the major cost factor in the DNA Sequencing type application such as the HaploSeq approach, is the cost of sequencing itself. As the method disclosed herein targets only exons (1-2% of the genome), the cost of obtaining chromosome-span haplotype can be reduced by over 20-30 fold. Second, the Whole-exome HaploSeq approach provides information on the most interpretable variants—the ones that harbor in the coding “exonic” and nearby regions of the genome. Furthermore, this computational approach can be used not only on single nucleotide variants (SNVs) as shown in the examples below, but also extended to other type of variants such as small indels and structural variations such as insertions, deletions, inversions, and translocations. These factors enable a more practical and an affordable variant of HaploSeq and opens up several applications

### Uses and Applications

There are many applications of the method and kits disclosed herein.

In some examples, they can be used for diploid sequencing of genomic elements of interest. Diploid sequencing allows genotyping, long-range or full-range haplotyping, 3D genome analysis of genomic elements (e.g. 3D organization of exomes), and other applications such as distinguishing between pseudo genomic elements (e.g. pseudo exons), calling structural variants in genomic elements (e.g. exon fusions or gene fusions, etc.).

In other examples, the method and kits can be used for chromosome-spanning haplotyping of those genomic elements of interest. Obtaining a haplotype in an individual is useful for a number of reasons. First, haplotypes are increasingly used as a means to detect disease associations. In addition, they are useful clinically in predicting outcomes for donor-host matching in organ transplantation and. Second, in genes that show compound heterozygosity, haplotypes provide information as to whether two deleterious variants are located on the same or different alleles, greatly impacting the prediction of whether inheritance of these variants are deleterious. In complex genomes such as humans, compound heterozygosity may involve genetic or epigenetic variations at non-coding cis-regulatory sites located far from the genes they regulate, underscoring the importance of obtaining chromosome-span haplotypes. Third, haplotypes from groups of individuals have provided information on population structure, and the evolutionary history of the human race. Lastly, widespread allelic imbalances in gene expression suggest that genetic or epigenetic differences between alleles may contribute to quantitative differences in expression. An understanding of haplotype structure will therefore be critical for delineating the mechanisms of variants that contribute to these allelic imbalances and for advancing personalized medicine.

The exome is the part of the genome formed by exons, the sequences which when transcribed remain within the mature RNA after introns are removed by RNA splicing. It consists of all DNA that is transcribed into mature RNA in cells of any type. The exome of the human genome consists of roughly 180,000 exons constituting about 1% of the total genome, or about 30 megabases of DNA (Ng et al., 2009, Nature 461 (7261): 272-276). Though comprising a very small fraction of the genome, mutations in the exome are thought to harbor 85% of mutations that have a large effect on diseases (Choi et al., 2009, Proc Natl Acad Sci USA 106 (45): 19096-19101). Exome haplotypes are important for determining the genetic basis of many genetic conditions and disorders.

Chromosome-spanning haplotypes have applications in non-invasive prenatal diagnostics (NIPD) and constructing ancestry. Conventional ways of generation of chromosome-spanning haplotypes are expensive, as they require whole-genome DNA sequencing, which is very costly and time consuming, and related haplotype phasing. The method disclosed herein provides an alternative in which one can target exons and still obtain chromosome-spanning haplotypes. Therefore, this invention allows one to obtain and use chromosome-spanning haplotypes in a cheaper and a more practical way.

For one, non-invasive fetal genome sequencing requires maternal haplotype information (Kitzman, et al. Sci Transl Med 4, 137ra76 (2012)). In this regard, the longer the maternal haplotype, the better the accuracy of fetal sequencing from maternal plasma. Ideally, generating chromosome-span maternal haplotypes will allow the most accurate sequencing of fetus from maternal plasma. By generating chromosome-span haplotypes at an affordable cost, the method disclosed herein therefore can generate the most accurate fetal sequencing from maternal plasma. Specifically, one can generate a maternal haplotype structure (from maternal blood sample or other sources) followed by whole-genome sequencing of the maternal plasma to reflect whole-genome fetal information. Alternatively, one can use targeted approaches such as exome sequencing on the maternal plasma to obtain exome sequencing of the fetus. In this regard, one can even target a set of actionable fetal genes or coding regions from maternal plasma. Regardless of targeted or whole-genome approaches on the fetus, a chromosome-span haplotype of the maternal genome is a critical input. Therefore, the method disclosed herein offers an affordable solution for a wide-variety of targeted and whole-exome sequencing opportunities of the fetus from maternal plasma.

Second, it has been shown that longer haplotype information can reveal recent ancestry of human population (Schiffels et al., Nat Genet 46, 919-25 (2014)). Therefore, by doing whole-exome HaploSeq or similar typing of other genomic elements of interest of many individuals across human population, one can decipher population structure as well as recent ancestral information (or pedigree) of human populations. In addition, ancestral information or population structure can also inform a great amount of detail in disease association analysis, pharmacogenomics and drug discovery. See e.g., Tewhey et al. Nat Rev Genet 12, 215-23 (2011).

Third, haplotype information can help to identify de novo mutations in an individual and therefore the method disclosed herein can be used in this case as well.

Organ transplantation will also benefit from haplotypes at the MHC and KIR locus. However, as genes outside this locus might potentially play a role in transplantation biology, whole-exome HaploSeq and similar typing of other genomic elements of interest could be useful.

Besides whole-exome HaploSeq application, whole-exome proximity ligation datasets can be useful for many other applications, including sequencing or genotyping, identifying gene fusions, de novo positioning of exons, identifying exonic structural variations, and understanding 3D structure of exomes. For instance, the proximity-ligation datasets can be used to perform genome scaffolding and consequently positioned several un-defined regions of the genome (Kaplan et al., Nat Biotechnol 31, 1143-7 (2013) and Burton et al., Nat Biotechnol 31, 1119-25 (2013)). In a similar manner, undefined and uncharacterized exons in a genome can be positioned de novo using Whole-Exome proximity-ligation datasets. As a consequence, one can identify exonic structural variations, exonic fusions and other structural variations in the genome. Using the 3D structure of exons, one can also delineate the relationship between spatial localization of genes/exons and their expression patterns-a key biological question in understanding functional regulation of genome.

In addition to using Whole-exome haploseq data for haplotype phasing, one can also use this data for exome-based variant calling and genotyping purpose. For example, the inventors used BWA Mem software to align the HaploSeq data to a reference genome followed by GATK pipeline to achieve variant calls and genotype information. Furthermore, it has been demonstrated that Hi-C/HaploSeq data can be used for genome assembly and for better understanding of the genome's repeat structure. Similarly, as Whole-exome HaploSeq reveals three-dimensional information on exons, it can be used for de novo assembly of exons, structural variation identifications such as gene fusions and translocations, haplotype phasing and genotyping. In sum, the reduced cost of the approach disclosed herein and the set of wide applications clearly provide the method of this invention a competitive advantage in the genomics market space.

### Kits

This invention further provides kits containing reagents for performing the above-described methods. Such kits can be used for applications including but not limited to genotyping, haplotyping, gene fusions, and 3D analyses of exomes. To that end, one or more of the reaction components for the methods disclosed herein can be supplied in the form of a kit for use. In one embodiment, the kit comprises a fixation agent, one or more restriction enzymes, a ligase, a set of probes that are complementary to sequences of the discontinuous genomic elements of interest (such as exonic sequences) in the one or more chromosomes, and are labeled with an affinity tag, and an agent capable of binding to the affinity tag. In others embodiments, the kit can include one or more other reaction components. In such a kit, an appropriate amount of one or more reaction components is provided in one or more containers or held on a substrate.

Examples of additional components of the kits include, but are not limited to, one or more components selected from the group consisting of a cell lysis buffer, one or more restriction enzyme reaction buffers, a hybridization buffer, extension nucleotides, a DNA polymerase, a protease, adaptors, blocking oligonucleotides, an RNAse inhibitor, reagents for sequencing, one or more cells, PCR primers. The kit may also include one or more of the following components: supports, terminating, modifying or digestion reagents, osmolytes, and an apparatus for detection. In some embodiments, the extension nucleotides can be labeled with an affinity tag.

The reaction components used can be provided in a variety of forms. For example, the components (e.g., enzymes, probes and/or primers) can be suspended in an aqueous solution or as a freeze-dried or lyophilized powder, pellet, or bead. In the latter case, the components, when reconstituted, form a complete mixture of components for use in an assay. The kits of the invention can be provided at any suitable temperature. For example, for storage of kits containing protein components or complexes thereof in a liquid, it is preferred that they are provided and maintained below 0° C., preferably at or below −20° C., or otherwise in a frozen state.

A kit may contain, in an amount sufficient for at least one assay, any combination of the components described herein. In some applications, one or more reaction components may be provided in pre-measured single use amounts in individual, typically disposable, tubes or equivalent containers. With such an arrangement, a proximity-ligation assay can be performed by adding a target nucleic acid, or a sample or cell containing the target nucleic acid, to the individual tubes directly. The amount of a component supplied in the kit can be any appropriate amount and may depend on the target market to which the product is directed. The container(s) in which the components are supplied can be any conventional container that is capable of holding the supplied form, for instance, microfuge tubes, microtiter plates, ampoules, bottles, or integral testing devices, such as fluidic devices, cartridges, lateral flow, or other similar devices.

The kits can also include packaging materials for holding the container or combination of containers. Typical packaging materials for such kits and systems include solid matrices (e.g., glass, plastic, paper, foil, micro-particles and the like) that hold the reaction components or detection probes in any of a variety of configurations (e.g., in a vial, microtiter plate well, microarray, and the like). The kits may further include instructions recorded in a tangible form for use of the components.

### Definitions

As disclosed herein, a number of ranges of values are provided. It is understood that each intervening value, to the tenth of the unit of the lower limit, unless the context clearly dictates otherwise, between the upper and lower limits of that range is also specifically disclosed. Each smaller range between any stated value or intervening value in a stated range and any other stated or intervening value in that stated range is encompassed within the invention. The upper and lower limits of these smaller ranges may independently be included or excluded in the range, and each range where either, neither, or both limits are included in the smaller ranges is also encompassed within the invention, subject to any specifically excluded limit in the stated range. Where the stated range includes one or both of the limits, ranges excluding either or both of those included limits are also included in the invention.

The term “about” generally refers to plus or minus 10% of the indicated number. For example, “about 10%” may indicate a range of 9% to 11%, and “about 1” may mean from 0.9-1.1. Other meanings of “about” may be apparent from the context, such as rounding off, so, for example “about 1” may also mean from 0.5 to 1.4.

The term “biological sample” refers to a sample obtained from an organism (e.g., patient) or from components (e.g., cells) of an organism. The sample may be of any biological tissue, cell(s) or fluid. The sample may be a “clinical sample” which is a sample derived from a subject, such as a human patient. Such samples include, but are not limited to, saliva, sputum, blood, blood cells (e.g., white cells), amniotic fluid, plasma, semen, bone marrow, and tissue or fine needle biopsy samples, urine, peritoneal fluid, and pleural fluid, or cells therefrom. Biological samples may also include sections of tissues such as frozen sections taken for histological purposes. A biological sample may also include a substantially purified or isolated protein, membrane preparation, or cell culture.

A “nucleic acid” refers to a DNA molecule (e.g., a genomic DNA), an RNA molecule (e.g., an mRNA), or a DNA or RNA analog. A DNA or RNA analog can be synthesized from nucleotide analogs. The nucleic acid molecule can be single-stranded or double-stranded, but preferably is double-stranded DNA.

The term “labeled nucleotide” or “labeled base” refers to a nucleotide base attached to a marker or tag, wherein the marker or tag comprises a specific moiety having a unique affinity for a ligand. Alternatively, a binding partner may have affinity for the marker or tag. In some examples, the marker includes, but is not limited to, a biotin, a histidine marker (i.e., 6His), or a FLAG marker. For example, dATP-Biotin may be considered a labeled nucleotide. In some examples, a fragmented nucleic acid sequence may undergo blunting with a labeled nucleotide followed by blunt-end ligation. The term “label” or “detectable label” are used herein, to refer to any composition detectable by spectroscopic, photochemical, biochemical, immunochemical, electrical, optical or chemical means. Such labels include biotin for staining with labeled streptavidin conjugate, magnetic beads (e.g., Dynabeads™), fluorescent dyes (e.g., fluorescein, texas red, rhodamine, green fluorescent protein, and the like), radiolabels (e.g., ³H, ¹²⁵I, ³⁵S, ¹⁴C, or ³²P), enzymes (e.g., horse radish peroxidase, alkaline phosphatase and others commonly used in an ELISA), and calorimetric labels such as colloidal gold or colored glass or plastic (e.g., polystyrene, polypropylene, latex, etc.) beads. The labels contemplated in the present invention may be detected or isolated by many methods.

“Affinity binding molecules” or “specific binding pair” herein means two molecules that have affinity for and bind to each other under certain conditions, referred to as binding conditions. Biotins and streptavidins (or avidins) are examples of a “specific binding pair,” but the invention is not limited to use of this particular specific binding pair. In many embodiments of the present invention, one member of a particular specific binding pair is referred to as the “affinity tag molecule” or the “affinity tag” and the other as the “affinity-tag-binding molecule” or the “affinity tag binding molecule.” A wide variety of other specific binding pairs or affinity binding molecules, including both affinity tag molecules and affinity-tag-binding molecules, are known in the art (e.g., see U.S. Pat. No. 6,562,575) and can be used in the present invention. For example, an antigen and an antibody, including a monoclonal antibody, that binds the antigen is a specific binding pair. Also, an antibody and an antibody binding protein, such as Staphylococcus aureus Protein A, can be employed as a specific binding pair. Other examples of specific binding pairs include, but are not limited to, a carbohydrate moiety which is bound specifically by a lectin and the lectin; a hormone and a receptor for the hormone; and an enzyme and an inhibitor of the enzyme.

As used herein, the term “oligonucleotide” refers to a short polynucleotide, typically less than or equal to 300 nucleotides long (e.g., in the range of 5 and 150, preferably in the range of 10 to 100, more preferably in the range of 15 to 50 nucleotides in length). However, as used herein, the term is also intended to encompass longer or shorter polynucleotide chains. An “oligonucleotide” may hybridize to other polynucleotides, therefore serving as a probe for polynucleotide detection, or a primer for polynucleotide chain extension.

“Extension nucleotides” refer to any nucleotide capable of being incorporated into an extension product during amplification, i.e., DNA, RNA, or a derivative if DNA or RNA, which may include a label.

The term “chromosome” as used herein, refers to a naturally occurring nucleic acid sequence comprising a series of functional regions termed genes that usually encode proteins. Other functional regions may include microRNAs or long noncoding RNAs, or other regulatory elements. These proteins may have a biological function or they directly interact with the same or other chromosomes (i.e., for example, regulatory chromosomes).

The term “genomic element” refers to a genomic nucleic acid sequence of interest. In general, such an element includes a defined sequence or a sequence substantially homologous to a defined sequence (e.g., a probe) to a degree sufficient to permit hybridization with a targeting element under the hybridization conditions employed. As used herein sequences “substantially homologous” refer to nucleic acid sequences that are identical or that share a very high homology with each other, such as, for example, at least 80%, 81%, 82%, 83%, 84%, 85%, 86%, 87%, 88%, 89%, 90%, 91%, 92%, 93%, 94%, 95%, 96%, 97%, 98% or 99% homology and that are found in the same genome.

The term “genome” refers to any set of chromosomes with the genes they contain. For example, a genome may include, but is not limited to, eukaryotic genomes and prokaryotic genomes. The term “genomic region” or “region” refers to any defined length of a genome and/or chromosome. Alternatively, a genomic region may refer to a complete chromosome or a partial chromosome. Further, a genomic region may refer to a specific nucleic acid sequence on a chromosome (i.e., for example, an open reading frame and/or a regulatory gene).

The term “regulatory element” as used herein, refers to any nucleic acid sequence that affects activity status of another genomic element. Examples include, but are not limited, to promoters, enhancer, repressors, insulators, boundary elements, origin of DNA replication, telomere, and/or centromere.

The term “regulatory gene” as used herein, refers to any nucleic acid sequence encoding a protein, wherein the protein binds to the same or different nucleic acid sequence thereby modulating the transcription rate or otherwise affecting the expression level of the same or different nucleic acid sequence.

A “variant” of a nucleotide is defined as a nucleotide sequence which differs from a reference oligonucleotide by having deletions, insertions and substitutions. These may be detected using a variety of methods (e.g., sequencing, hybridization assays etc.).

The term “fragments” refers to any nucleic acid sequence that is shorter than the sequence from which it is derived. Fragments can be of any size, ranging from several megabases and/or kilobases to only a few nucleotides long. Experimental conditions can determine an expected fragment size, including but not limited to, restriction enzyme digestion, sonication, acid incubation, base incubation, microfluidization etc.

The term “fragmenting” refers to any process or method by which a compound or composition is separated into smaller units. For example, the separation may include, but is not limited to, enzymatic cleavage (i.e., for example, transposase-mediated fragmentation, restriction enzymes acting upon nucleic acids or protease enzymes acting on proteins), base hydrolysis, acid hydrolysis, or heat-induced thermal destabilization.

The term “fixing,” “fixation” or “fixed” refers to any method or process that immobilizes any and all cellular processes. A fixed cell, therefore, accurately maintains the spatial relationships between intracellular components at the time of fixation. Many chemicals are capable of providing fixation, including but not limited to, formaldehyde, formalin, or glutaraldehyde.

The term “crosslinking” or “crosslink” refers to any stable chemical association between two compounds, such that they may be further processed as a unit. Such stability may be based upon covalent and/or non-covalent bonding. For example, nucleic acids and/or proteins may be cross-linked by chemical agents (i.e., for example, a fixative) such that they maintain their spatial relationships during routine laboratory procedures (i.e., for example, extracting, washing, centrifugation etc.)

The term “ligated” as used herein, refers to any linkage of two nucleic acid sequences usually comprising a phosphodiester bond. The linkage is normally facilitated by the presence of a catalytic enzyme (i.e., for example, a ligase) in the presence of co-factor reagents and an energy source (i.e., for example, adenosine triphosphate (ATP)).

The term “restriction enzyme” refers to any protein that cleaves nucleic acid at a specific base pair sequence.

As used herein “bait” or “probe” sequences refer to synthetic long oligonucleotides or oligonucleotides derived from (e.g., produced using) synthetic long oligonucleotides that are complementary to target nucleic acids of interest. In certain embodiments, the set of bait sequences is derived from oligonucleotides synthesized in a microarray and cleaved and eluted from the microarray. In other embodiments, the bait sequences are produced by nucleic acid amplification methods, e.g., using human DNA or pooled human DNA samples as the template.

Bait sequences preferably are oligonucleotides between about 70 nucleotides and 1000 nucleotides in length, more preferably between about 100 nucleotides and 300 nucleotides in length, more preferably between about 130 nucleotides and 230 nucleotides in length and more preferably still are between about 150 nucleotides and 200 nucleotides in length. For selection of exons and other short targets, preferred bait sequence lengths can be oligonucleotides of about 40 and 1000, e.g., 100 to about 300 nucleotides, more preferably about 130 to about 230 nucleotides, and still more preferably about 150 to about 200 nucleotides. For selection of targets that are long compared to the length of the capture baits, such as genomic regions, preferred bait sequence lengths are typically in the same size range as the baits for short targets mentioned above, except that there is no need to limit the maximum size of bait sequences for the sole purpose of minimizing targeting of adjacent sequences. Methods to prepare longer oligonucleotides for bait sequences are well known in the art.

In some embodiments, the bait sequences in the set of bait sequences can be RNA molecules. RNA molecules preferably are used as bait sequences since RNA-DNA duplex is more stable than a DNA-DNA duplex, and therefore provides for potentially better capture of nucleic acids. RNA bait sequences can be synthesized using any method known in the art, including in vitro transcription. If RNA is synthesized using biotinylated UTP, single stranded biotin-labeled

RNA bait molecules are produced. In preferred embodiments, the RNA baits correspond to only one strand of the double-stranded DNA target. As those skilled in the art will appreciate, such RNA baits are not self-complementary and are therefore more effective as hybridization drivers. In certain embodiments, RNase-resistant RNA molecules are synthesized. Such molecules and their synthesis are well known in the art.

As used herein, the term “hybridization” or “binding” refers to the pairing of complementary (including partially complementary) polynucleotide strands. Hybridization and the strength of hybridization (e.g., the strength of the association between polynucleotide strands) is impacted by many factors well known in the art including the degree of complementarity between the polynucleotides, stringency of the conditions involved affected by such conditions as the concentration of salts, the melting temperature (Tm) of the formed hybrid, the presence of other components, the molarity of the hybridizing strands and the G:C content of the polynucleotide strands. When one polynucleotide is said to “hybridize” to another polynucleotide, it means that there is some complementarity between the two polynucleotides or that the two polynucleotides form a hybrid under high stringency conditions. When one polynucleotide is said to not hybridize to another polynucleotide, it means that there is no sequence complementarity between the two polynucleotides or that no hybrid forms between the two polynucleotides at a high stringency condition.

The term “antibody” refers to immunoglobulin produced in animals in response to an immunogen (antigen). It is desired that the antibody demonstrates specificity to epitopes contained in the immunogen. The term “polyclonal antibody” refers to immunoglobulin produced from more than a single clone of plasma cells; in contrast “monoclonal antibody” refers to immunoglobulin produced from a single clone of plasma cells.

The terms “specific binding” or “specifically binding” when used in reference to the interaction of any compound with a nucleic acid or peptide wherein the interaction is dependent upon the presence of a particular structure (i.e., for example, an antigenic determinant or epitope). For example, if an antibody is specific for epitope “A”, the presence of a protein containing epitope A (or free, unlabelled A) in a reaction containing labeled “A” and the antibody will reduce the amount of labeled A bound to the antibody.

### EXAMPLES

### Example 1

In this example, it was examined whether whole-exome haplotype phasing can be achieved using datasets obtained from simulated genome proximity-ligation assays (such as TCC or Hi-C or in-situ Hi-C). More specifically, in order to show that whole-exome haplotype phasing is feasible, data from a Hi-C whole-genome proximity-ligation experiment for chromosome 1 from GM12878 cells were obtained. Then, fragments that contained exonic regions in at least one of the two sequenced read pairs were retained. Accordingly, this dataset represented a simulated whole-exome proximity-ligation dataset.

The data was then simulated using the algorithm described above and the simulated data was used to check its ability to phase exonic SNVs into a single haplotype structure. For that, two metrics were defined-Completeness defines the length of haplotype block compared to the length of chromosome and Resolution defines fraction of exonic variants phased in the chromosome. It was found that regardless of read-length chosen, complete haplotypes were achieved and that longer read lengths help generate higher-resolution haplotypes, e.g., 250 bp paired ends.

As shown in FIGS. 3a-e, chromosome-span complete haplotypes were successfully generated regardless of the chosen read length for sequencing (FIG. 3a-e). These simulated results also indicate that longer read lengths generated higher resolution haplotypes (as measured by the fraction of exonic variants phased), and therefore are more preferable for Whole-exome HaploSeq (FIG. 3e). These results demonstrate that data from a whole-genome proximity-ligation can be used generate chromosome-span haplotypes using the approach disclosed in this invention.

### Example 2

In this example, it was examined whether whole-exome haplotype phasing can be achieved using real datasets obtained from exome capture proximity-ligation.

More specifically, exome capture was performed on proximity-ligation data from GM12878 cells and followed by sequencing in the manner described above. The exome capture protocol was internally optimized for fragment length, blocking primers, and oligonucleotide probe binding. As shown in FIG. 4, three whole-exome proximity-ligation libraries were generated. Two of these libraries were using a single enzyme (NcoI or XbaI) while a third was generated using a pooled collection of 6 base cutting enzymes (HindIII, NcoI, XbaI, and BamHI-labeled as “multi-enzyme”). After capture and sequencing, it was found that these libraries had clear enrichment of exonic sequences (FIG. 4b). They were then sequenced to generate ˜50-70 million read pairs for each library (FIG. 4b).

First these datasets were used to show the sequencing or genotyping capability of Whole-exome proximity-ligation assays. To that end, inventors were able to identify ˜60-65% of exonic variants from each of these datasets individually. Interestingly, the multi-enzyme dataset (Figure c-i) genotyped more variants than NcoI dataset (FIG. 4c (i), despite having only half the sequencing read depth. FIGS. 4c (ii)-(iv) show the results of whole-genome genotyping from NcoI (ii), Multi-enzyme (iii) combined dataset (iv). These results indicate that multi-enzyme data can be more useful for genotyping and potentially haplotyping or de novo assembly applications when compared to single enzyme dataset.

By combining each of the three datasets together, over 85% of variants were identified (FIG. 4c-i). To check the accuracy of the identified variants, the genotyping results were compared with previously identified genotype calls for GM12878 cells (International HapMap, C. et al. Nature 449, 851-61 (2007) and Genomes Project, C. et al. A map of human genome variation from population-scale sequencing. Nature 467, 1061-73 (2010)). It was observed that for both homozygous and heterozygous variant calls, the accuracy of the method of this invention was very high->99% for heterozygous and >95% for homozygous. Despite most of the data from Whole-exome proximity-ligation libraries tending to occupy exons, a significant fraction may target non-exonic regions that are spatially proximal with exonic regions. Using this, the inventors genotyped 52% of all variants (exonic and non-exonic) in the genome (FIG. 4c-ii-iv). The results demonstrate that Whole-exome HaploSeq dataset can generate high accuracy exonic and whole-genome genotyping or sequencing.

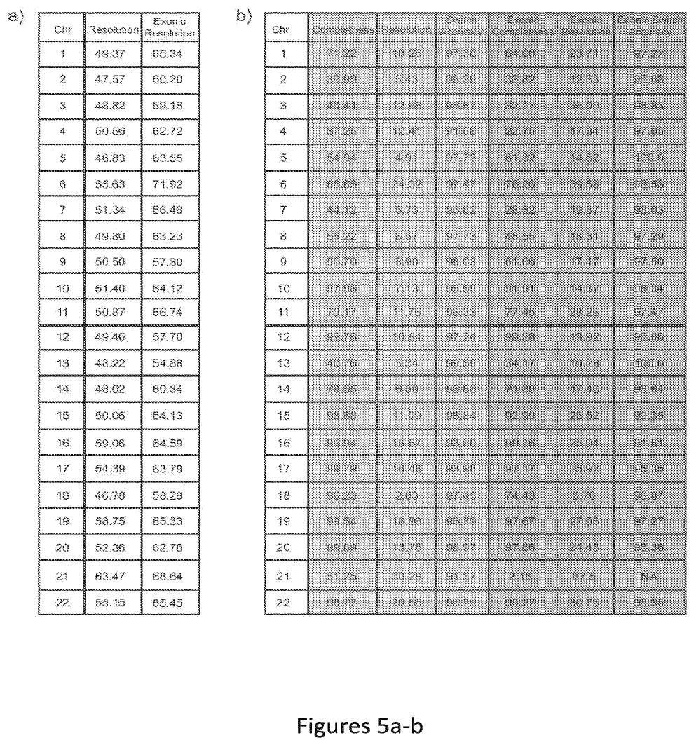

Next, the haplotyping capabilities of Whole-exome proximity-ligation assays were checked using the combined dataset. To do this, a graph where exons are considered as edges and link exons was built based on the data. Then a maxcut based algorithm was used to construct best possible exonic phasing, as predicted by exon links in the data. Using this strategy, phase was resolved successfully for over 50% of all variants (SNVs) and more importantly >65% of exonic variants (FIG. 5a). While >50% of variants (or 65% of exonic variants) were phased, the variants may not belong to the same haplotype block. Specifically, variants can be phased across multiple haplotype blocks, giving rise to “incomplete” phasing. To check the ability to generate a complete chromosome-span haplotype, only results from the longest haplotype-most variant phased (MVP) block were considered (FIG. 5b).

It was found that chromosome-span haplotypes were successfully generated for most of the chromosomes-in particular smaller chromosomes such as 15-22. For smaller chromosomes, the method tended to phase majority of chromosome (50-70%) of variants into a single haplotype block. The same results held true if only exonic variants were considered (FIG. 5b—orange). To this end, while 65% of exonic variants were phased in any haplotype block, ˜20% of them belonged to the MVP block on average. This indicates that for many chromosomes, chromosome-span complete haplotypes were successfully generated at a resolution of ˜20%. Furthermore, by comparing the haplotype identifications to previously identified haplotype calls for GM12878 cells (International HapMap, C. et al. Nature 449, 851-61 (2007) and Genomes Project, C. et al. A map of human genome variation from population-scale sequencing. Nature 467, 1061-73 (2010)), the accuracy was found to be ˜97% on average.

While the subsection shown in FIG. 5a describes the phasing results across all the haplotype block, the most useful one is the block with the most variant phased (i.e., MVP). In previous HaploSeq, the MVP block was chromosome-span and phased most of the variant (>80%). In Whole-exome HaploSeq here, the MVP block (FIG. 5b) was chromosome-span haplotypes for most of the chromosomes-especially for the small chromosomes. Because only the exonic regions that align with restriction-enzyme cut-sites were targeted, resolution in the MVP block was on the lower side. To this end, very high accuracy was achieved. The orange section in FIG. 5b (columns 2-4) describes MVP metrics based on all the SNVs while the green sections (columns 5-7) describes that based on the exonic SNVs. The accuracy and completeness were similar across both these definitions, with resolution higher on the exonic SNVs, as expected.

Together, the above results show that using Whole-exome proximity-ligation assays, one can generate comprehensive and accurate genotypes and that these datasets can be used to generate complete chromosome-span accurate haplotypes for the chromosomes.

### Example 3

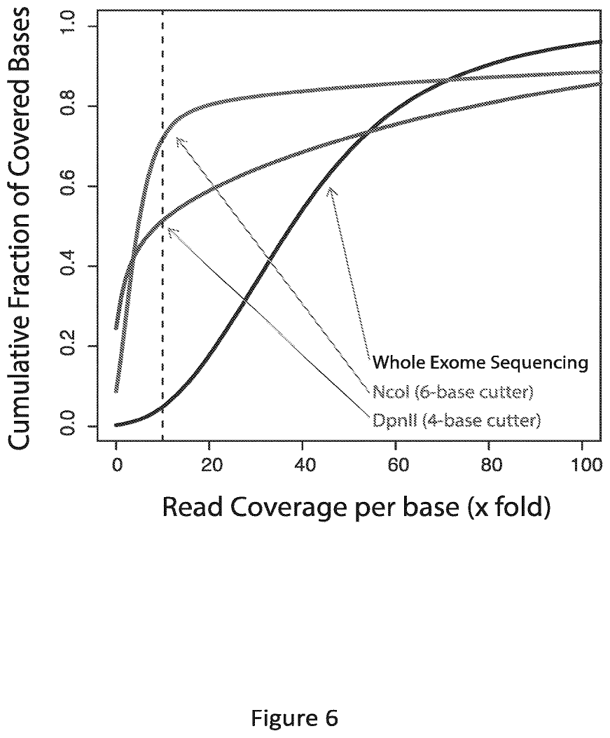

In this example, assays were carried out to examine the effect of restriction enzyme choice in terms of the number of bases that are covered and phased. Briefly, three libraries were generated, using the exome sequencing protocols and the Whole-Exome Haploseq approach described above. For that, NcoI (a 6-base cutting enzyme) and DpnI (a 4-base cutting enzyme) were used. The results are shown in FIG. 6. It was found that when each library was sequenced to an average coverage of 44×, 96% of bases are covered at ≥10× in the whole exome sequencing sample. However, if a 6 base cutter was used, this was only about 30% of bases that were covered at or above 10×. In the case of the 4-base cutting enzyme, this was improved to 50%. These results again indicate that multi-enzyme data can be more useful for genotyping and potentially haplotyping or de novo assembly applications as compared to single enzyme dataset.

The foregoing examples and description of the preferred embodiments should be taken as illustrating, rather than as limiting the present invention as defined by the claims. As will be readily appreciated, numerous variations and combinations of the features set forth above can be utilized without departing from the present invention as set forth in the claims. Such variations are not regarded as a departure from the scope of the invention, and all such variations are intended to be included within the scope of the following claims. All references cited herein are incorporated by reference herein in their entireties.

What is claimed is:

1. A method for typing and assembling discontinuous genomic elements, comprising
obtaining a plurality of genomic DNA fragments or a genomic sequence data of one or more chromosomes;
obtaining a plurality of element sequence reads of the genomic elements from the genomic DNA fragments or the genomic sequence data, and
assembling the plurality of element sequence reads to genotype and construct a long-range or chromosome-span haplotype for the one or more chromosomes.

2. The method of claim 1, wherein the plurality of genomic DNA fragments are obtained using a proximity-ligation based technique.

3. The method of claim 1 or 2, wherein the discontinuous genomic elements are selected from the group consisting of genes, exons, introns, untranslated regions, protein domain-coding sequences, gene fusions, transcription factor binding sites, promoters, enhancers, silencers, conserved elements, miRNA-coding sequences, miRNA binding sites, splice sites, splicing enhancers, splicing silencers, structure variants, common SNPs, UTR regulatory motifs, post translational modification sites, and common elements.

4. The method of claim 2 or 3, wherein the plurality of genomic DNA fragments are obtained by a process comprising:
providing a cell that contains a set of chromosomes having genomic DNA;
incubating the cell or the nucleus thereof with a fixation agent for a period of time to allow crosslinking of the genomic DNA in situ and thereby to form crosslinked genomic DNA;
fragmenting the crosslinked genomic DNA;
ligating the crosslinked and fragmented genomic DNA to form a proximally ligated complex;
shearing the proximally ligated complex to form proximally-ligated DNA fragments; and
obtaining a plurality of the proximally-ligated DNA fragments to form a library thereby obtaining the plurality of genomic DNA fragments.

5. The method of claim 4, wherein the fragmenting step is carried out by restriction enzyme digestion with one or more enzymes.

6. The method of claim 5, wherein the digestion is carried out with two or more different enzymes.

7. The method of claim 5 or 6, wherein at least one of the enzymes is a 4-cutter or 6-cutter.

8. The method of any one of claims 1-7, wherein the plurality of element sequence reads are obtained from the genomic DNA fragments by a process comprising:
hybridizing the plurality of genomic DNA fragments with a set of probes to form a hybridization mixture;
separating probes that are hybridized to isolate a subgroup of the genomic DNA fragments, and
sequencing the isolated genomic DNA fragments to generate a plurality of sequence reads thereby obtaining the plurality of element sequence reads,
wherein the probes comprise sequences complementary to sequences of the discontinuous genomic elements in the one or more chromosomes.

9. The method of claim 8, further comprising amplifying the isolated genomic DNA fragments before the sequencing step.

10. The method of any one of claims 8-9, wherein the set of probes comprises an affinity tag on each probe.

11. The method of claim 10, wherein the affinity tag is a biotin molecule or a hapten.

12. The method of claim 11, wherein the separating step comprising contacting the hybridization mixture with an agent that binds to the affinity tag.

13. The method of claim 12, wherein the agent is an avidin molecule, or an antibody that binds to the hapten or an antigen-binding fragment thereof.

14. The method of any one of claims 8-13, wherein the probes are attached to a support.

15. The method of claim 14, wherein the support is a microarray.

16. The method of claim 14 or 15, wherein the support comprises a planar support comprising one or more substrate materials selected from glass, silicas, metals, teflons, and polymeric materials.

17. The method of any one of claims 14-16, wherein the support comprises a mixture of beads, each bead having one or more probes bound thereto.

18. The method of claim 17, wherein the mixture of beads comprises one or more substrate materials selected from nitrocellulose, glass, silicas, teflons, metals, and polymeric materials.

19. The method of any one of claims 8-18, wherein the discontinuous genomic elements are exons or protein domain-coding sequences, and the probes are cDNA probes or RNA probes.

20. The method of any one of claims 3-19, further comprising isolating the cell nucleus from the cell before the incubating step.

21. The method of any one of claims 3-20, further comprising purifying genomic DNA before the fragmenting step.

22. The method of any one of claims 3-21, wherein the fixation agent comprises formaldehyde, glutaraldehyde, formalin, or a combination thereof.

23. The method of any one of claims 8-22, wherein the sequencing step is carried out using next generation sequencing (NGS).

24. The method of claim 1, wherein the genomic sequence data comprises a plurality of sequence reads for genes, exons, introns, untranslated regions, protein domain-coding sequences, gene fusions, transcription factor binding sites, promoters, enhancers, silencers, conserved elements, miRNA-coding sequences, miRNA binding sites, splice sites, splicing enhancers, splicing silencers, structure variants, common SNPs, UTR regulatory motifs, post translational modification sites, and common elements.

25. The method of any one of claims 1-24, wherein the chromosomes are from a cell of an organism.

26. The method of claim 25, wherein the organism is a eukaryote.

27. The method of claim 26, wherein the organism is a fungus, a plant, or an animal.

28. The method of claim 27, where the organism is a mammal or a mammalian embryo.

29. The method of claim 28, wherein the organism is a human.

30. The method of claim 28, wherein the chromosomes are derived from a human embryo.

31. The method of any one of claims 1-30, wherein the assembling is carried out using a maxcut algorithm, with or without population-based imputation.

32. The method of any one of claims 1-31, further comprising genotyping or variant calling.

33. A kit for performing the method of any one of claims 1-32, comprising a fixation agent;
one or more restriction enzymes;
a ligase;
a set of probes that are complementary to sequences of the discontinuous genomic elements in the one or more chromosomes, and are labeled with an affinity tag, and
an agent capable of binding to the affinity tag.

34. The kit of claim 33, further comprising one or more components selected from the group consisting of a cell lysis buffer, one or more restriction enzyme reaction buffers, a hybridization buffer, extension nucleotides, a DNA polymerase, a protease, adaptors, blocking oligonucleotides, an RNAse inhibitor, and reagents for sequencing.

35. The kit of claim 34, wherein at least one of the extension nucleotides is labeled with an affinity tag.
